(19) United States
(12) Patent Application Publication
(10) Pub. No.: US 2005/0209632 A1
(43) Pub. Date: Sep. 22, 2005

(54) Filtering devices

(76) Inventor: Michael J. Wallace, Bellaire, TX (US)

Correspondence Address: FULBRIGHT & JAWORSKI L.L.P., 600 CONGRESS AVE., SUITE 2400, AUSTIN, TX, 78701, US

(21) Appl. No.: 11/035,388

(22) Filed: Jan. 13, 2005

Related U.S. Application Data
(60) Provisional application No. 60/536,387, filed on Jan. 14, 2004.

(51) Int. Cl. A61M 29/00

(52) U.S. Cl. 606/200

(57) ABSTRACT

Filters suited for use in, for example, blood vessels in an animal body. In one embodiment, a filter that includes two or more independent struts; and a filter element having a filter portion and strut-engaging members, each strut-engaging member being releasably engaged with an independent strut. Other embodiments and methods of making the present filters are included.

### CROSS-REFERENCE(S) TO RELATED APPLICATION(S)

This application claims priority to U.S. Provisional Patent Application Ser. No. 60/536,387, filed Jan. 14, 2004, the entire contents of which are expressly incorporated by reference.

### BACKGROUND OF THE INVENTION

1. Field of the Invention

The invention relates generally to the field of medical devices. More particularly, it relates to filters that may be placed internally in a patient and, under certain conditions, (a) modified to remove a filtering element of the filter, or (b) removed completely.

2. Description of Related Art

Filters, such as vascular filters, may be used for a variety of therapeutic applications, including capturing thrombus (i.e., blood clots), or for distal protection during a vascular procedure. Filters are generally either permanent or temporary. Permanent filters are implanted for patients that require a filter for more than 14 days. Fourteen days roughly approximates the time before which the points where the filter contact the vessel wall become covered by endothelial cells that attach the filter to the vessel wall. If an attempt is made to remove the filter after this point, severe damage to the patient's vessel or other anatomy may result.

Some filters can serve as either a temporary or a permanent filter, depending on the need of the patient. These filters generally involve a filtering mechanism that is either removable or modifiable. Examples of filters with removable or modifiable filtering mechanisms include U.S. Pat. Nos. 6,241,746, 6,267,776, 6,506,205, 6,517,559, and 6,582,447, all of which are incorporated by reference. Among these filters with removable filtering mechanisms, a stent or stent-like anchoring structure is left behind in the vessel when the filtering mechanism is removed. The inventor has discovered that such a structure (i.e., a stent or stent-like anchoring mechanism) may produce unwanted stress on the vessel. Additionally, vessel wall interactions with metallic stents can result in the formation of intimal hyperplasia, which could result in luminal narrowing or occlusion.

### SUMMARY OF THE INVENTION

The present filters may be used as permanent filters, temporary filters that may be removed completely under appropriate conditions, or temporary filters that may be rendered temporary by removing a filtering element and leaving behind two or more independent struts that will put little or no pressure on the vessel wall. As a result, the present filters are simple, versatile, and less likely to produce unwanted stress on vessels than previous filters.

In one embodiment, a filter is provided that has two or more independent struts, and a filter element that has a filter portion and strut-engaging members. In this embodiment, each strut-engaging member is releasably engaged with an independent strut.

In another embodiment, a filter is provided that is configured for use in a patient, and that comprises six struts and a filter element. The filter element has a filter portion that includes three main members that share a common apex, a V-shaped segment interconnecting each pair of main members, and a secondary member extending from each V-shaped segment. In this embodiment, the filter element also includes a strut-engaging member extending from each main member at an angle of less than 90 degrees, and a strut-engaging member extending from each secondary member at an angle of less than 90 degrees. In this embodiment, each strut-engaging member is releasably engaged with a strut, and the struts (i) are not interconnected, and (ii) are not interconnectable by any structure other than the filter element.

Additional embodiments of the present filters, and details associated with those embodiments, are set forth below. Also set forth below are methods for making the present filters.

### BRIEF DESCRIPTION OF THE DRAWINGS

The following drawings illustrate by way of example and not limitation. The use of identical reference numerals does not necessarily indicate an identical structure. Rather, the same reference numeral may be used to indicate a similar feature or a feature with similar functionality. Reference numerals should not be used to construe the claims.

FIG. 1A is a perspective view of one of the present filters.

FIG. 1B is a perspective view of the filter shown in FIG. 1A, the only difference being that the filter element shown in FIG. 1B includes segments that are curved rather than straight.

FIG. 2 is a top view of the filter shown in FIG. 1A.

FIG. 3 is a partial front view of one of the present independent struts that has a hollow portion. The view shows only a portion of one of the present strut-engaging members releasably engaged with the depicted independent strut.

FIG. 4 is a bottom view of the independent strut and portion of a strut-engaging member shown in FIG. 3.

FIG. 5 is a partial front view of one of the present independent struts that has a groove. The view shows only a portion of one of the present strut-engaging members releasably engaged with the depicted independent strut.

FIG. 6 is a bottom view of the independent strut and portion of a strut-engaging member shown in FIG. 5.

FIG. 7 is an enlarged view of what is depicted in FIG. 6, and shows how one pair of the present strut-engaging member retention portions may function.

FIG. 8 is a partial front view of one of the present independent struts that has a groove and two retention flaps. The view shows only a portion of one of the present strut-engaging members releasably engaged with the depicted independent strut.

FIG. 9 is a bottom view of the independent strut and portion of a strut-engaging member shown in FIG. 8.

FIG. 10 is an enlarged view of what is depicted in FIG. 9, and shows how the depicted retention flaps and strut-engaging member retention portions may function.

FIG. 11A is a partial front view of the one of the present independent struts in which an opening has been cut or otherwise positioned in order to accept an opening-engaging segment of one of the present strut-engaging members.

FIGS. 11B, 11C, and 11D are partial front views of suitable configurations for the present opening-engaging segments of the present strut-engaging members.

FIG. 12 is a partial view showing an angle between one of the present independent struts and one of the present main members.

FIG. 13 is a partial view showing a secondary angle between one of the present independent struts and one of the present secondary members.

FIGS. 14-16 are a series of views showing stages of one manner of removing a filter element of one of the present filters.

FIG. 17 is a partial view of one of the present independent struts that is configured to releasably engage one of the present strut-engaging members by virtue of the hollow portion (shown partially in cross-section) provided in the strut and the bend in the strut-engaging member.

FIG. 18 is a partial view of one of the present independent struts that is configured to releasably engage one of the present strut-engaging members by virtue of the hollow portion (shown partially in cross-section) provided in the strut and an adhesive in contact with both a portion of the strut-engaging member and a portion of the wall defining the hollow portion.

FIG. 19 is a partial view of one of the present independent struts that is configured to releasably engage one of the present strut-engaging members by virtue of the hollow portion and crimps (shown partially in cross-section) provided in the strut.

FIGS. 20A-20D are a series of views showing stages additional to those shown in FIGS. 14-16 of one manner of removing a filter element of one of the present filters.

FIGS. 21A-21D are a series of views showing stages of one manner of removing one of the present filters in which the independent struts are releasably engaged with (but not yet disengaged from) the strut-engaging members.

### DESCRIPTION OF ILLUSTRATIVE EMBODIMENTS

The terms “comprise” (and any form of comprise, such as “comprises” and “comprising”), “have” (and any form of have, such as “has” and “having”), and “include” (and any form of include, such as “includes” and “including”) are open-ended linking verbs. As a result, a device that “comprises,” “has,” or “includes” one or more elements possesses those one or more elements, but is not limited to possessing only those one or more elements. Likewise, an element of a device that “comprises,” “has,” or “includes” one or more features possesses those one or more features, but is not limited to possessing only those one or more features.

Thus, and by way of example, a filter “comprising” two or more independent struts; and a filter element having a filter portion and strut-engaging members, where each strut-engaging member is releasably engaged with an independent strut has, but is not limited to having only, the recited features. That is, the filter possesses at least the recited features, but does not exclude other features that are not expressly recited.

The terms “a” and “an” are defined as one or more than one unless this disclosure explicitly requires otherwise.

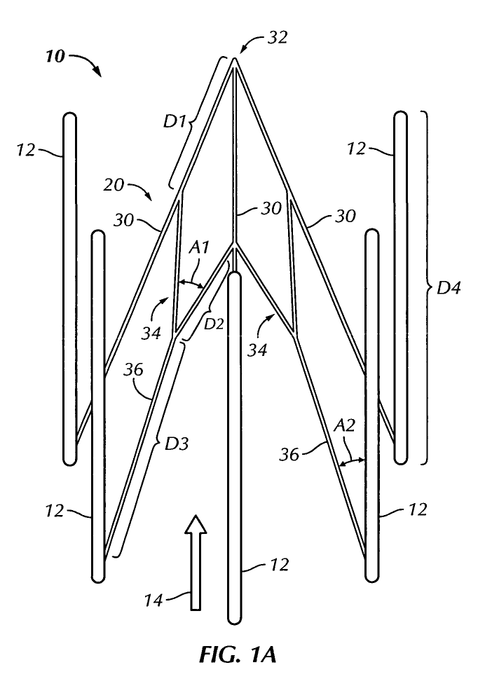

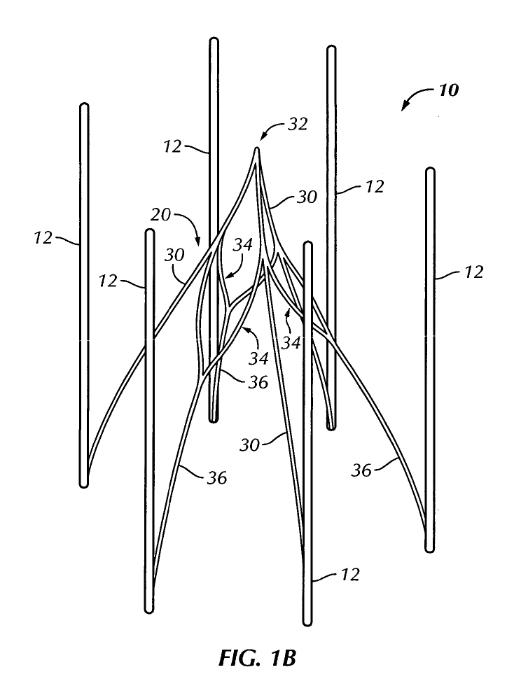

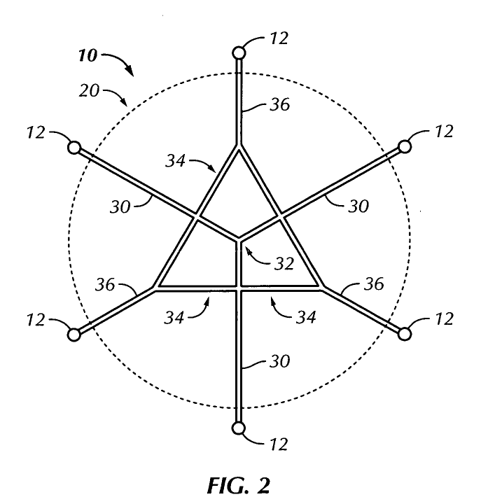

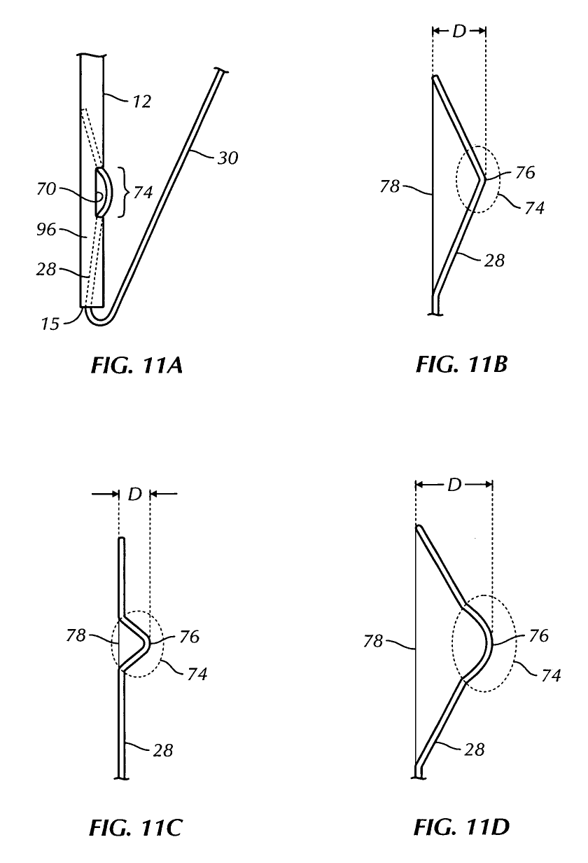

One version of the present filters is depicted in FIGS. 1A and 2. Filter 10 includes two or more independent struts 12 and a filter element 20. Filter element 20 includes a filter portion 24 (designated generally by dashed lines in FIG. 2) and strut-engaging members 28 (not visible in FIGS. 1A and 2, but visible in FIGS. 11A-11D and 12, for example). Each independent strut 12, of which there are 6 in this embodiment, is configured to releasably engage a strut-engaging member 28. Such configurations of independent struts 12 are shown and described below. For simplicity, FIG. 1A does not illustrate the sixth independent strut shown in FIG. 2.

Orientation members 112 of U.S. Pat. No. 6,517,559 are not “releasably engaged” with filter legs 102 (see FIG. 12 of the '559 patent) because the two are either permanently connected together (by welding or the like), or are formed from the same wire. An “independent strut” is defined as a strut that is not linked or otherwise connected to any other independent strut except by one of the present filter elements (or by a vessel wall following incorporation). Accordingly, vertical struts 18 in U.S. Pat. No. 6,436,121 are not “independent struts” because they are linked to each other by hollow tubular member 14.

The independent struts of the present filters are preferably straight, as illustrated in the figures. They may, however, be provided with a slight curve to better fit a given application. One or more barbs 16 (which may be unidirectional, as shown) may be provided in any suitable location along the length of one or more (and up to all) of the independent struts of any of the present filters (see FIG. 1A). Such barbs can help to maintain the position of the independent struts in a hollow body structure.

In the embodiment shown in FIGS. 1A and 2, filter portion 24 of filter element 20 includes main members 30, which share a common apex 32; and V-shaped segments 34 interconnecting each pair of main members 30 (one of the V-shaped segments 34 depicted in FIG. 2 is not depicted in FIG. 1A for simplicity). V-shaped segments 34 serve to strengthen filter element 20 by linking main members 30 to each other at a location other than apex 32. A strut-engaging member 28 extends from each main member 30. In this embodiment, filter portion 24 also includes a secondary member 36 extending from the caudal end (which is pointed in substantially the opposite direction of apex 32) of each V-shaped segment 34. In this embodiment, a strut engaging-member 28 extends from each second member 36. As shown in the figures, V-shaped segments 34 may be symmetrically positioned such that secondary members 36 are centered between the two main members that the V-shaped segments connect.

The features of the depicted embodiment of the present filters that contribute the most to the filtering function of filter portion 24 are the apex 32, V-shaped segments 34, and the portions of main members 30 that extend between apex 32 and V-shaped segments 34. Nevertheless, filter portion 24 is designated in FIGS. 1A and 2 as including secondary members 36 and the remaining portions of main members 30 because the same contribute at least somewhat to the filtering function of filter portion 24.

Filter 10 is suited for use in a patient (e.g., by implantation), such as a human or other animal. Filter 10 may be sized to fit in, for example, the inferior or superior vena cava, or in any other vessel or hollow body structure in which a filter is needed. The direction that blood flow may be oriented through filter 10 is shown by arrow 14 in FIG. 1A.

The size of the present filters will vary depending on the application. For use in some vessels, the diameter of the base of, for example, filter 10 (the base diameter being the distance between opposing independent struts 12 in a delivered state) may be between 30 to 40 millimeters (mm) and the height of filter 10 (from the bottom of a strut-engaging member 28 to apex 32) should be approximately equal to the base diameter for an inferior vena cava application. The thicknesses of independent struts 12 and the wires (should wires be used) that make up the various features of filter element 20 will be based on the outward fixation forces determined to hold filter 10 in place.

Exemplary dimensions of the features of filter 10 adapted for use in the inferior vena cava include the following: the length of main member 30 may be 4 centimeters (cm); D1 (see FIG. 1A), which is the length of the segment of main member 30 between apex 32 and the location at which one arm of V-shaped segment 34 connects to main member 30, may be 1 cm; D2, which is the length of each of the arms of V-shaped segment 34 (in certain embodiments, both arms may have the same length, although it will be possible to vary their lengths somewhat in certain applications), may be 1 cm; D3, which is the length of secondary member 36, may be 2 cm; D4, which is the length of independent strut 12, may be 4 cm; A1, which is the angle separating the two arms of a given V-shaped segment 34, may be 60 degrees; and A2, which is the angle separating secondary member 36 from independent strut 12, may be 45 degrees.

FIG. 1A shows that the various features of filter element 20, which may be used in one version of the present filters, can be straight. Alternatively, those same features may be curved as shown in FIG. 1B. Such curves may help the filter element, and therefore the entire filter in some cases, to more easily be compressed when loaded into a delivery catheter or other delivery system, when being retrieved, or when packaged for sale. The curves may also help to more evenly distribute the stresses that result from compression of the filter element.

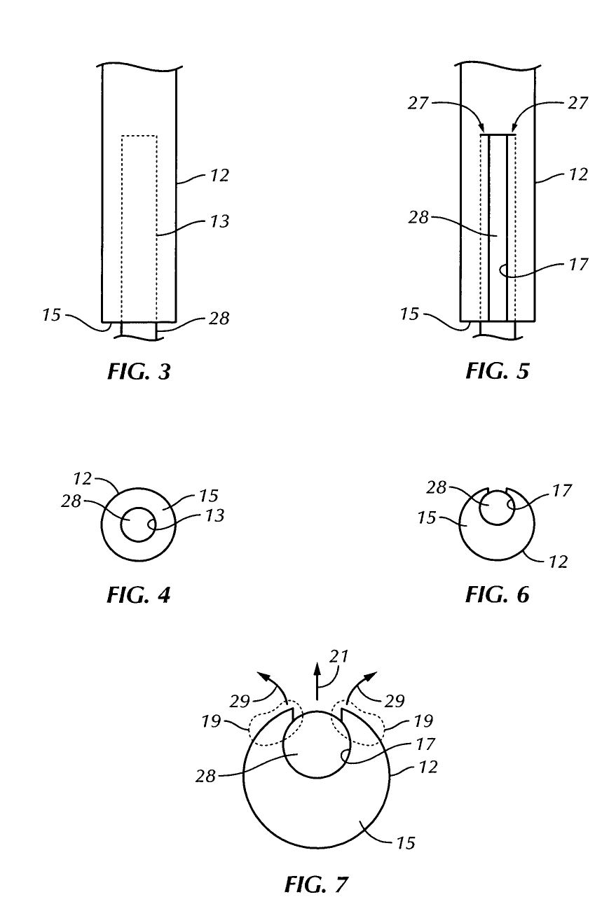

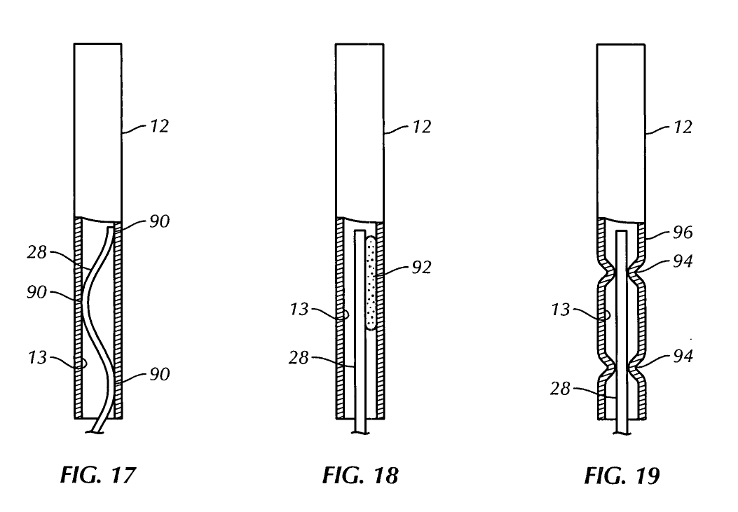

The independent struts 12 of the present filters can be configured to releasably engage the present strut-engaging members 28 in a variety of ways. One such configuration is shown in FIGS. 3 and 4. FIG. 3 shows that independent strut 12 may be provided with a hollow portion 13 (shown in dashed lines) in which at least a portion of a strut-engaging member 28 (which may extend from a main member 30 or a secondary member 36) may be slidably positionable (e.g., one manner of releasable engagement). Hollow portion 13 may begin at one end 15 of independent strut 12 and extend toward, but not necessarily reach, the other end (not shown) of independent strut 12. The length of hollow portion 13 will be dictated by the mechanism (examples of which are discussed below and shown in the figures) used to releasably engage the independent strut 12 of which hollow portion 13 is a part and the strut-engaging member 28 corresponding to that independent strut. The tolerances between the outer diameter of strut-engaging members 28 and the diameter of hollow portion 13 (which may also be characterized as the inner diameter of independent strut 12) may be sufficiently small that friction will exist between the two that helps to keep them together and serves as the mechanism effecting the releasable engagement. A friction fit that provides releasable engagement may also be accomplished by bending strut-engaging member 28 as shown in FIG. 17 such that at least three points of contact 90 are created between the bent strut-engaging member and the wall defining hollow portion 13.

As shown in FIG. 19, another form of a friction fit that provides releasable engagement may be accomplished using one or more crimps 94 of the wall 96 of independent strut 12 that defines hollow portion 13. That is, independent strut 12 may include a wall 96 that defines hollow portion 13, and wall 96 (which may also be described as an interior wall) may include one or more crimps 94 that releasably engage strut-engaging member 28 due to the friction between the portion or portions of the inside of wall 96 that contact strut-engaging member 28.

As an alternative to a friction-fit, an adhesive 92 may be used as shown in FIG. 18 to maintain the releasable engagement between an independent strut 12 having a hollow portion 13 and the strut-engaging member 28 positioned in hollow portion 13. Adhesive 92 may be biocompatible and possess time-sensitive biodegradability that will not permit the strut-engaging member 28 from disengaging independent strut 12 until a chosen time has elapsed. The time may be based on the amount of time that is predicted for incorporation. For example, the bond created by the adhesive could be strong for 2-4 weeks so that filter 10 in its entirety could be retrieved if desired, but after 4 weeks (or the chosen time) the bond will have degraded enough that the strut-engaging member is able to slide freely out of the hollow portion in which it is positioned, allowing the strut-engaging member to be disengaged from the independent strut.

Another configuration of independent strut 12 suitable for releasably engaging a strut-engaging member is illustrated in FIGS. 5-7. FIG. 5 shows that a groove 17 may be provided in a portion of independent strut 12 in which at least a portion of a strut-engaging member 28 (which may extend from a main member 30 or a secondary member 36) may be slidably positionable. Groove 17 may begin at one end 15 of independent strut 12 and extend toward, but not necessarily reach, the other end (not shown) of independent strut 12. Groove 17 may have any suitable length. FIG. 6 is a bottom view of the independent strut 12 and portion of strut-engaging member 28 shown in FIG. 5.

FIG. 7 is an enlargement of the view in FIG. 6, and illustrates that by providing independent strut 12 with groove 17, independent strut 12 may—as a result—be provided with two strut-engaging member retention portions 19 (outlined generally in dashed lines). These two strut-engaging member retention portions 19 may be sufficiently thin and pliable that, as an alternative to sliding strut-engaging member 28 out of groove 17, strut-engaging member 28 may be pulled in the direction of arrow 21 in order to release strut-engaging member 28 from engagement with independent strut 12. The pulling action will bend back (e.g., in the directions indicated by arrows 29) strut-engaging member retention portions 19, allowing strut-engaging member 28 to be released from engagement with independent strut 12. The amount of force required to remove strut-engaging member 28 in this fashion should be no more than could possibly cause damage a vessel wall in which independent strut 12 is at least partially entrained (e.g., by endothelial tissue). Slits 27 may be provided in independent strut 12—extending from the exterior of independent strut 12 to the inner wall that defines groove 17—at the location where groove 17 terminates, so that strut-engaging member retention portions 19 may bend as shown by arrows 29 from end 15 of independent strut 12 all the way up to slits 27. A combination of pulling in direction 21 and sliding strut-engaging member 28 out of groove 17 may also be used to release strut-engaging member 28 from engagement with this version of independent strut 12.

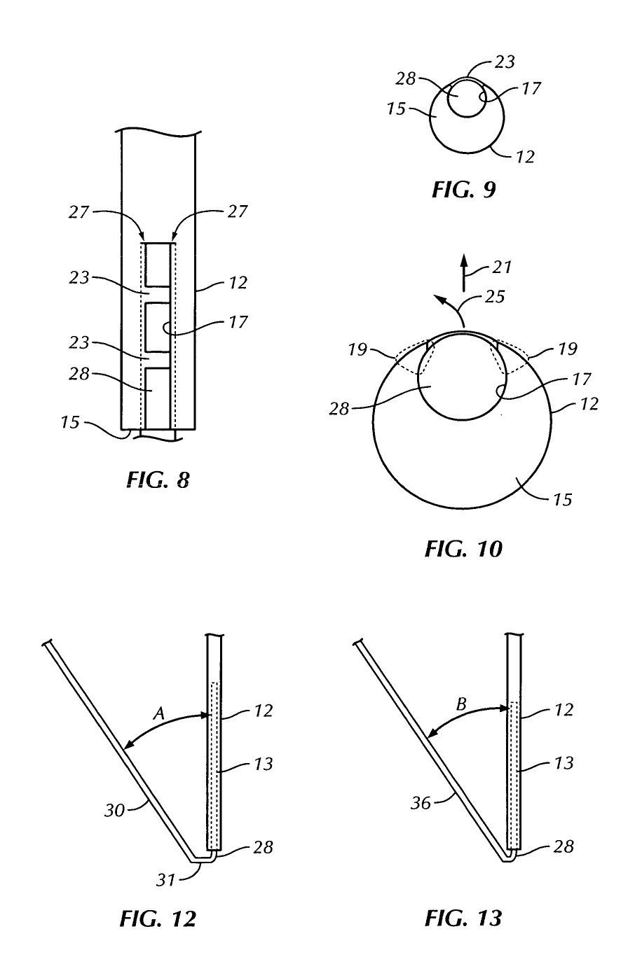

Yet another configuration of independent strut 12 suitable for releasably engaging a strut-engaging member is illustrated in FIGS. 8-10. FIG. 8 shows that groove 17 may be supplemented by one or more retention flaps 23 that extend from one exterior side of groove 17 to the other. By pulling strut-engaging member 28 (which may extend from a main member 30 or a secondary member 36) in the direction of arrow 21, retention flap or flaps 23 may be bent back in the direction of arrow 25 (FIG. 10) while strut-engaging member retention portions 19 bend back in the directions shown in FIG. 7, thus allowing strut-engaging member 18 to be released from engagement with independent strut 12. FIG. 9 is a bottom view of the independent strut 12 and portion of strut-engaging member 28 shown in FIG. 8.

Still another configuration of independent strut 12 suitable for releasably engaging a strut-engaging member is illustrated in FIG. 11A. This figure shows that, in addition to having a hollow portion (not labeled), independent strut 12 may include an opening 70 that is cut into its wall 96 such that the hollow portion communicates with the exterior of independent strut 12. Opening 70 may have any suitable shape, such as rectangular, oval, or the like. Strut-engaging member 28 extending from main member 30 (or secondary member 36, as the case may be) may be provided with a curved opening-engaging segment 74 along its length that is configured to extend at least partially through opening 70. As shown in FIG. 11B, opening-engaging segment 74 may take the form of a simple bend. The distance D between the tip 76 of bend 74 and the back 78 of strut-engaging member 28 should be greater than the inner diameter of the hollow portion of independent strut 12. As a result, a spring force will effectively exist in strut-engaging member 28 that forces bend 74 to extend at least partially through opening 70, as shown in FIG. 11A. Bend 74 will, in turn, encounter resistance from the bottom edge of groove 70 if strut-engaging member 28 is moved in the direction of end 15 of independent strut 12. Until that resistance is overcome, it will keep strut-engaging member 28 in place.

Another suitable shape for opening-engaging segment 74 is shown in FIG. 11C. There, opening-engaging segment 74 takes the form of a protrusion that projects from an otherwise straight strut-engaging member 28. Distance D for this version of opening-engaging member 74 should be the same as for the bend version.

FIG. 11D depicts another suitable shape for opening-engaging segment 74. In this version, opening-engaging segment takes the form of a protrusion—similar to what is shown in FIG. 11C—that projects from a bent strut-engaging member 28. In essence, the opening-engaging segment 74 depicted in FIG. 11D is a hybrid of those shown in FIGS. 11B and 11C. Distance D for this version of opening-engaging member 74 should be the same as for the bend version shown in FIG. 11B.

FIGS. 12 and 13 illustrate that an acute angle exists between the present strut-engaging members 28 and the members (either main or secondary) from which they extend. FIG. 12 is a partial view of an independent strut 12 having a hollow portion 13 (although a groove 17—with or without retention flap(s) 23—could alternatively be used) in which a strut-engaging member 28 is slidably positioned. Strut-engaging member 28 extends from main member 30, and an angle A exists between the two. As shown, angle A is less than 90 degrees. Such an angle A may exist between each main member 30 and strut-engaging member 28 extending from that main member. FIG. 12 also shows that such an angle may exist despite the presence of a 1-2 millimeter (mm) segment 31 that is oriented substantially perpendicular to strut-engaging member 28. In another embodiment, no such segment 31 exists.

FIG. 13 is another partial view of an independent strut 12 having a hollow portion 13 in which a strut-engaging member 28 is slidably positioned. In this figure, strut-engaging member 28 extends from secondary member 36, and a secondary angle B exists between the two. As illustrated, secondary angle B is less than 90 degrees. Such a secondary angle B may exist between each secondary member 36 and strut-engaging member 28 extending from that secondary member. No segment 31 is shown, although one may be provided in the same manner illustrated in FIG. 12.

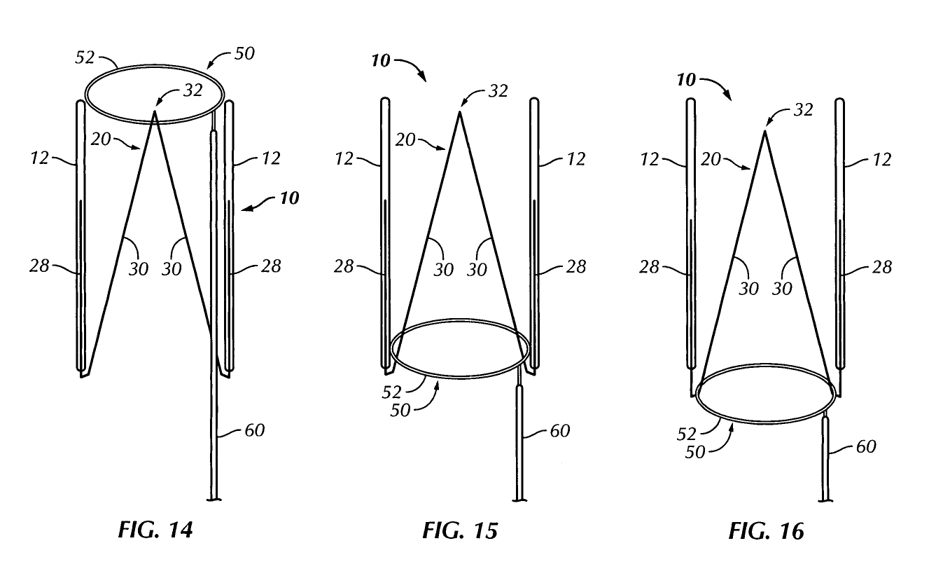

The present filters may be delivered to a target site using any suitable technique, such as the Seldinger technique. Once one of the present filters is in place, using filter 10 as an example, filter element 20 may be removed by using, for example, a snare. FIGS. 14-16 are a series of views showing a portion (for simplicity) of filter element 20 of filter 10, and how the same may be removed using a snare 50.

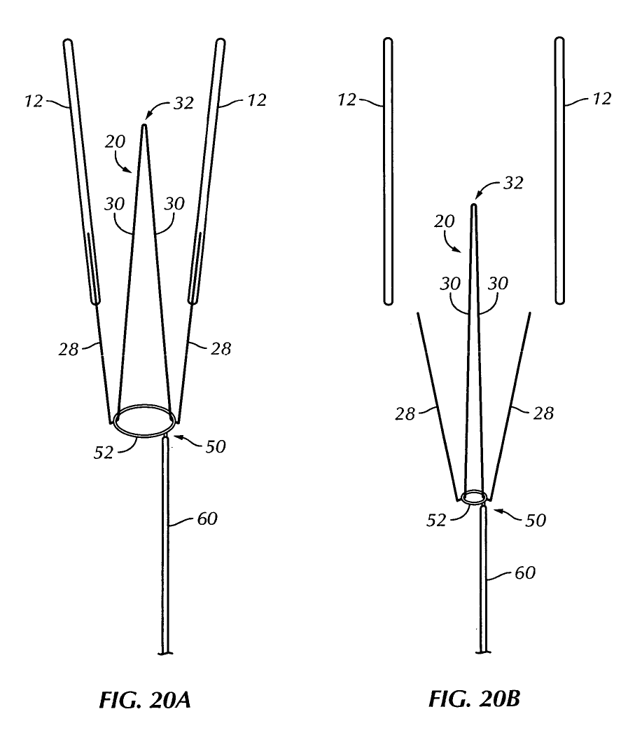

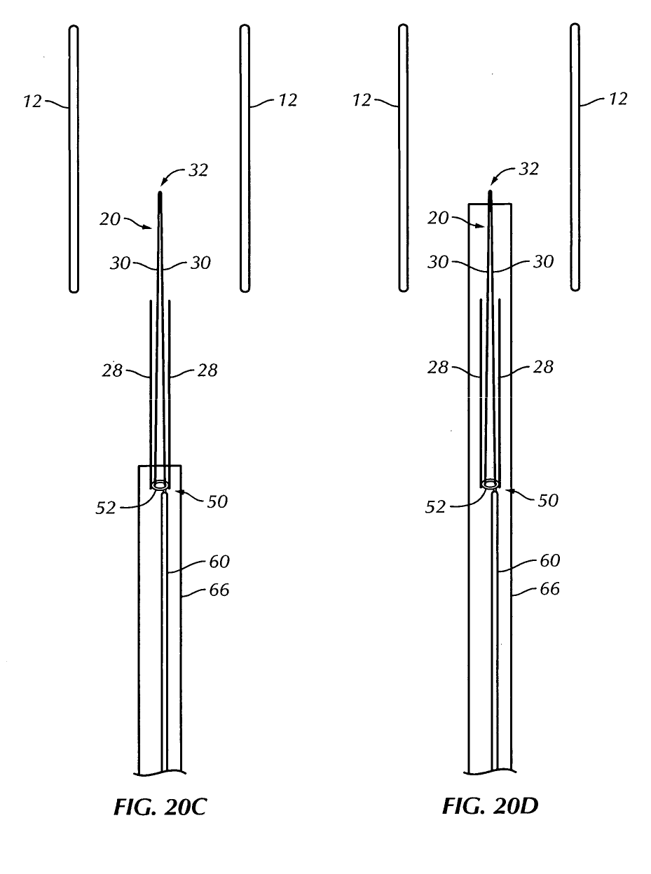

The distal end of catheter 60, which houses snare 50, may be directed to a location downstream of apex 32 of filter element 20. Snare 50—such as an AMPLATZ snare—may be advanced sufficiently out of catheter 60 to expose loop 52. Loop 52 may then be positioned over apex 32, as shown in FIG. 14. As shown in FIG. 15, catheter 60 and snare 50 may then be withdrawn such that loop 52 slides over the members of filter element 20. FIG. 16 shows that as loop 52 of snare 50 comes into contact with the portion of filter element 20 where the members (e.g., main and/or secondary members) meet the strut-engaging members 28 extending from them, snare 50 and catheter 60 may continue to be withdrawn such that the strut-engaging members 28 are disengaged from their releasably engagement with independent struts 12. After this, the base of filter element 20 (i.e., the widest portion of filter element 20) may be reduced in size to facilitate removal of filter element 20 through a recovery sheath. This may be accomplished by reducing the size of loop 52 of snare 50. Such a size reduction may result in pulling all members (both main and secondary) of filter element 20 tightly together. Pulling snare 50 into catheter 60 will close loop 52 of the snare, thus pulling the members of filter element 20 tightly together. This scenario is shown in stages in FIGS. 20A-20D, where the recovery sheath is labeled 66.

The removal illustrated in FIGS. 14-16 may occur at virtually any stage of incorporation of independent struts 12 into a wall of a vessel or other hollow body structure. Even when independent struts 12 become entrained in endothelial tissue, it should be possible to either (a) slide the strut-engaging members 28 out of the grooves or hollow portions provided in independent struts 12, (b) pull and slide strut-engaging members 28 out of the grooves provided in independent struts 12, or (c) pull strut-engaging members 28 out of the grooves provided in independent struts 12. Because independent struts 12 are not interconnected to each other after removal of filter element 20, the hollow body structure in which they are incorporated undergoes no unnecessary stress.

Furthermore, the present independent struts 12 have less material than many prior filters. As a result, use of the present independent struts 12 will involve substantially less foreign metal that is retained in a vessel or other hollow structure and that would otherwise potentially produce intimal hyperplasia.

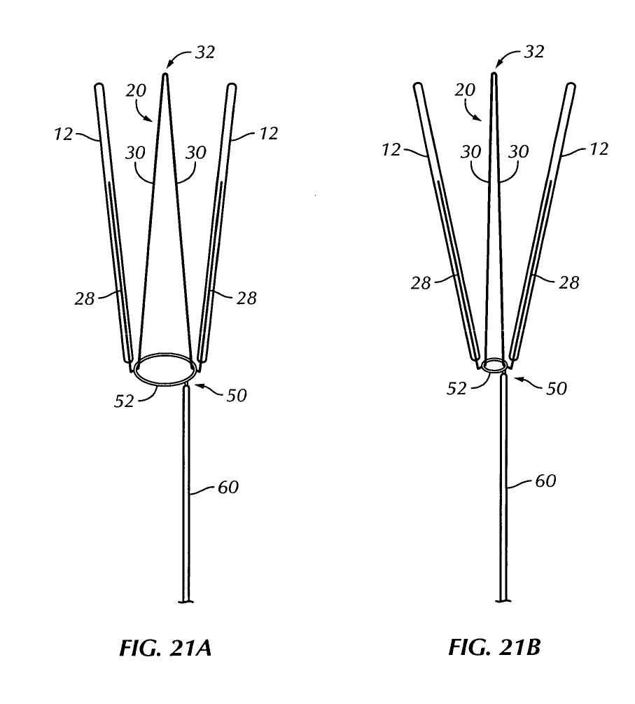

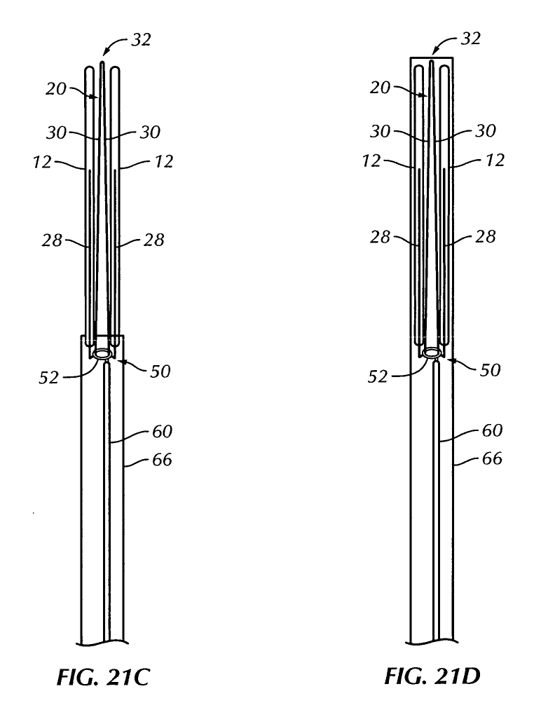

During use of filter 10, for example, if independent struts 12 have not incorporated into the vessel wall or other hollow body structure and filter removal is desired, the same process (discussed above) that can be used to remove filter element 24 can be used to remove filter 10 entirely. This follows because the mechanism by which strut-engaging members 28 are releasably engaged with independent struts 12 will be of sufficient force that the pairs of strut-engaging members and releasably engaged independent struts will move as a unit unless the independent strut is incorporated. The releasing force should be sufficient to hold the two together during filter placement and early retrieval but not too strong as to cause the independent struts to tear away from the vessel or other hollow body structure if incorporation has taken place. This allows entire filter 10 to be retrievable up to a time period of 2-4 weeks, or partially retrievable by removing only the filtering element 24 after incorporation. FIGS. 21A-21D show various stages of removal of a portion of filter 10 (for simplicity) into recovery sheath 66. The stages involve the same procedure described above for removing filter element 20.

The independent struts of the present filters, along with the various features of the present filter portions, may be formed of any suitable material, including the nickel-titanium alloy sold under the name NITINOL. Other non-ferrous metals (i.e., magnetic resonance imaging (MRI) compatible) may also be used.

The present filters may be manufactured by several techniques. Taking filter 10 as an example, one is to produce filter element 20, including strut-engaging members 28, from a single tube of metal by laser cutting. When the tube is made from a nickel-titanium alloy such as NITINOL, the alloy may be unheated and possess superelastic and kink-resistant properties. Alternatively, such an alloy may be positioned in a preferred shape and heat treated—as is well known to those of ordinary skill in the art—in order to utilize its thermal-memory properties. Another method of production is to weld or solder the individual features of filter element 20 together. Those individual elements may be made from a nickel-titanium alloy such as NITINOL and left untreated or heat-treated, as just described.

It should be understood that the present filters and methods of making them are not intended to be limited to the particular forms disclosed. Rather, they are to cover all modifications, equivalents, and alternatives falling within the scope of the claims. For example, although a filter with 6 independent struts has been illustrated, more or less independent struts may be used consistently with the present filters. As few as 2 independent struts or as many as 16, or any suitable number in between (including 3, 4, 5, 6, 7, 8, 9, 10, 11, 12, 13, 14, and 15), may be used consistently with the present filters. Furthermore, in embodiments of the present filters not shown in the figures, V-shaped segments 34 depicted in FIGS. 1A and 2 are not provided.

The claims are not to be interpreted as including means-plus- or step-plus-function limitations, unless such a limitation is explicitly recited in a given claim using the phrase(s) “means for” or “step for,” respectively.

1. A filter configured for use in a patient, comprising:
two or more independent struts; and
a filter element having a filter portion and strut-engaging members, each strut-engaging member being releasably engaged with an independent strut.

2. The filter of claim 1, where at least one of the independent struts includes a barb.

3. The filter of claim 1, where at least one of the independent struts includes a groove, and a strut-engaging member is slidably positioned in the groove.

4. The filter of claim 1, where each independent strut includes a groove, and each strut-engaging member is slidably positioned in the groove of an independent strut.

5. The filter of claim 4, where the filter portion includes (i) three main members that share a common apex and (ii) a V-shaped segment interconnecting each pair of main members; and a strut-engaging member extends from each main member.

6. The filter of claim 5, where the filter portion further includes a secondary member extending from each V-shaped segment; and a strut-engaging member extends from each secondary member.

7. The filter of claim 1, where the filter portion includes (i) three main members that share a common apex, and (ii) a V-shaped segment interconnecting each pair of main members; and a strut-engaging member extends from each main member.

8. The filter of claim 7, where the filter portion further includes a secondary member extending from each V-shaped segment; and a strut-engaging member extends from each secondary member.

9. The filter of claim 8, where an angle exists between each main member and the strut-engaging member extending from that main member, and each angle is less than 90 degrees.

10. The filter of claim 9, where a secondary angle exists between each secondary member and the strut-engaging member extending from that secondary member, and each secondary angle is less than 90 degrees.

11. The filter of claim 1, where the filter portion includes main members that share a common apex; and a strut-engaging member extends from each main member.

12. The filter of claim 11, where an angle exists between each main member and the strut-engaging member extending from that main member, and each angle is less than 90 degrees.

13. The filter of claim 1, where at least one of the independent struts includes a hollow portion, and a strut-engaging member is slidably positioned in the hollow portion.

14. The filter of claim 13, where at least one of the independent struts includes a hollow portion, and a strut-engaging member is (a) slidably positioned in the hollow portion and (b) releasably engaged with the hollow portion by virtue of friction.

15. The filter of claim 14, where the hollow portion is bordered by an interior wall, and the friction is the result of at least three contact points between the interior wall and the strut-engaging member.

16. The filter of claim 14, where the hollow portion is bordered by an interior wall, and the friction is the result of one or more crimps in the interior wall that contact the strut-engaging member.

17. The filter of claim 13, where at least one of the independent struts includes a hollow portion, and a strut-engaging member is (a) slidably positioned in the hollow portion and (b) releasably engaged with the hollow portion as a result of an adhesive.

18. The filter of claim 1, where at least one of the independent struts includes a wall, a hollow portion, and an opening in the wall; and a strut-engaging member includes an opening-engaging segment that extends at least partially through the opening.

19. The filter of claim 18, where the opening-engaging segment is a bend.

20. The filter of claim 18, where the opening-engaging segment is a protrusion.

21. A filter configured for use in a patient, comprising:
six struts; and
a filter element having:
a filter portion that includes three main members that share a common apex, a V-shaped segment interconnecting each pair of main members, and a secondary member extending from each V-shaped segment;
a strut-engaging member extending from each main member at an angle of less than 90 degrees; and
a strut-engaging member extending from each secondary member at an angle of less than 90 degrees;
where each strut releasably engages a strut-engaging member, and the struts (i) are not interconnected, and (ii) are not interconnectable by any structure other than the filter element.

22. The filter of claim 21, where at least one of the struts includes a barb.

23. The filter of claim 21, where at least one of the struts includes a groove, and one of the strut-engaging members is slidably positioned in the groove.

24. The filter of claim 21, where each strut includes a groove, and each strut-engaging member is slidably positioned in the groove of a strut.

25. The filter of claim 21, where at least one of the struts includes a hollow portion, and a strut-engaging member is (a) slidably positioned in the hollow portion and (b) releasably engaged with the hollow portion by virtue of friction.

26. The filter of claim 25, where the hollow portion is bordered by an interior wall, and the friction is the result of at least three contact points between the interior wall and the strut-engaging member.

27. The filter of claim 25, where the hollow portion is bordered by an interior wall, and the friction is the result of one or more crimps in the interior wall that contact the strut-engaging member.

28. The filter of claim 21, where at least one of the struts includes a hollow portion, and a strut-engaging member is (a) slidably positioned in the hollow portion and (b) releasably engaged with the hollow portion as a result of an adhesive.
